(19) United States
(12) Patent Application Publication
(10) Pub. No.: US 2004/0225232 A1
(43) Pub. Date: Nov. 11, 2004

(54) Sensor guide wire

(75) Inventors: Par von Malmborg, Uppsala (SE); Leif Smith, Uppsala (SE); Dan Akerfeldt, Uppsala (SE)

Correspondence Address: FOLEY AND LARDNER, SUITE 500, 3000 K STREET NW, WASHINGTON, DC, 20007, US

(73) Assignee: RADI MEDICAL SYSTEMS AB

(21) Appl. No.: 10/434,354

(22) Filed: May 9, 2003

(51) Int. Cl. A61B 5/00

(52) U.S. Cl. 600/585

(57) ABSTRACT

Core wire for a sensor guide wire assembly for intravascular measurements of physiological variables in a living body, the core wire (20, 30) is provided with different longitudinal sections each having a section diameter (φA-φG), and comprises an enlarged portion, where a sensor is adapted to be arranged, including a predetermined number of sections (C, D, E), one or many distal sections (F, G) positioned distally said enlarged portion and one or many proximal sections (A, B) positioned proximally said enlarged portion, wherein at least one of the sections of said enlarged portion has a larger diameter compared to the diameters of the distal and proximal sections. The ratio between the diameters (φF, φC) adjacent the enlarged portion is close to 1.

### FIELD OF THE INVENTION

The invention relates generally to sensors mounted on guide wires for intravascular measurements of physiological variables in a living body, in particular to the design of such guide wires, and more particularly to the design of the core wires in such guide wires.

### BACKGROUND OF THE INVENTION

Sensor and guide wire assemblies in which a sensor, adapted for measurements of physiological variables in a living body, such as blood pressure and temperature, is mounted at the distal end of a guide wire are known.

In U.S. Pat. No. 5,226,423, which is assigned to the assignee of the present patent specification, is disclosed one example of such a sensor and guide wire assembly, in which a sensor guide comprises a sensor element, an electronic unit, a signal transmitting cable connecting the sensor element to the electronic unit, a flexible tube having the cable and the sensor element disposed therein, a solid metal wire, which is disposed inside the sensor guide and extends along the entire length of the sensor guide inside the flexible tube, and a coil attached to the distal end of the solid metal wire. The solid metal wire, also known as a core wire, has been divided into a plurality of sections, and near the distal end of the sensor guide, the sensor element is positioned in one of these sections having an enlarged thickness. The entire contents of this patent are incorporated herein by reference for further details of such a guide wire arrangement.

A sensor and guide wire assembly, similar to that of U.S. Pat. No. 5,226,423 and also assigned to the assignee of the present patent specification, is shown in U.S. Pat. No. 6,142,958. According to U.S. Pat. No. 6,142,958, the core wire extends out from the distal end of the flexible tube, and a first coil is provided between the section having an enlarged thickness and the distal end of the flexible tube, while a second coil is attached to the distal end of the section having an enlarged thickness. The entire contents of this patent are incorporated herein by reference for further details of such a guide wire arrangement.

According to U.S. Pat. No. 5,226,423 and U.S. Pat. No. 6, 142,958 each of the plurality of core wire sections has a different thickness and thereby a different flexibility. Apparently, a large flexibility of the sensor guide is advantageous in that it allows the sensor guide to be introduced into small and tortuous vessels. It should, however, also be recognized that if the core wire is too flexible, it would be impossible to push the sensor guide forward into the vessels, i.e. the sensor guide must possess a certain “pushability”.

Furthermore, the sensor guide wire must be able to withstand the mechanical stress exerted on the core wire especially in sharp vessel bends. In U.S. Pat. No. 5,226,423 and U.S. Pat. No. 6, 142,958 no figures are given for the dimensions (e.g. the diameters) of the different sections of the core wire, but a sensor guide of this type which is manufactured and sold by the assignee of the present patent specification under the registered trademark PRESSUREWIRE® has a portion adjacent the proximal end of the enlarged section, which contains the sensor element, with a diameter of 130 μm and a portion adjacent the distal end of the enlarged section with a diameter of 90 μm.

Although this design of the known sensor guide wire has proven to work very well it can be improved.

### SUMMARY OF THE INVENTION

The object of the present invention is therefore to provide a core wire to be used in a sensor and guide wire assembly with such design and such dimensions that the sensor and guide wire assembly is provided with improved mechanical properties regarding flexibility and strength.

This object is achieved with a core wire according to the present invention.

According to the present invention it has been found that the mechanical strength of a sensor guide wire of the type described above is surprisingly sensitive to the dimensions of the different core wire portions distally and proximally of the enlarged portion accommodating the sensor element. In particular, tests have shown that the portion of the core wire that is adjacent the proximal end of this enlarged portion should have the same diameter as the portion of the core wire that is adjacent the distal end of the enlarged portion.

By following these findings, it has even more surprisingly been found that the diameter of the portion proximally of the enlarged portion can be decreased without deteriorating the strength of the core wire as a whole. In fact, extensive tests have shown that by reducing the diameter of the portion proximally of the enlarged portion by 20 μm, from 130 μm to 110 μm, and by increasing the diameter of the portion distally of the enlarged portion by 20 μm, from 90 μm to 110 μm, the strength of the sensor guide wire is enhanced by a factor of seven (7).

### BRIEF DESCRIPTION OF THE DRAWINGS

FIG. 1 is a cross-section of a sensor and guide wire assembly according to a conventional design.

FIG. 2 shows schematically a core wire according to a first preferred embodiment of the present invention.

FIG. 3 illustrates the behaviour in sharp bends of a core wire provided with a stiff portion.

FIG. 4 illustrates the behaviour in sharp bends of a wire without a stiff portion.

FIG. 5 shows schematically a core wire according to a second preferred embodiment of the present invention.

FIG. 6 is a cross-section of an enlarged section of a core wire, with a sensor element disposed therein.

### DETAILED DESCRIPTION OF PREFERRED EMBODIMENTS

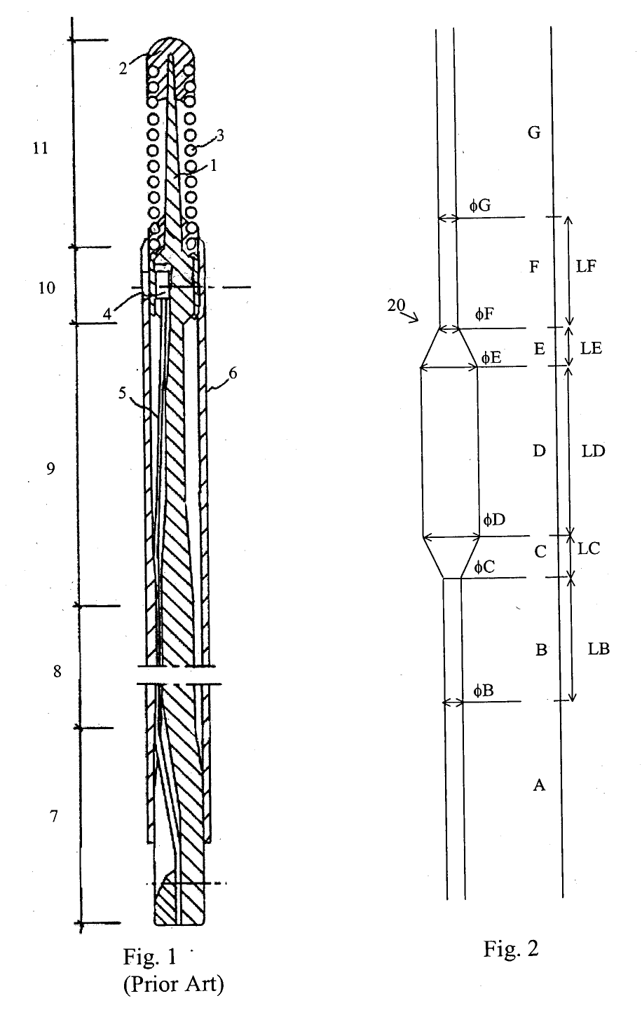

Now with reference to FIG. 1, a sensor guide wire according to the above-identified prior art comprises a core wire 1 whose distal end is provided with a dome-shaped tip 2, a coil 3 attached to the dome-shaped tip 2 and to an enlarged portion of the core wire 1, a sensor element 4 to which at least one signal transmitting cable 5 is connected, and an outer tube 6 which at least partly encloses the core wire 1.

The sensor and guide wire assembly of FIG. 1 has been divided into five sections, 7-11, where section 11 is the most distal section of the assembly, i.e. the section that is going to be inserted farthest into a vessel, and section 7 is the most proximal section. In a preferred embodiment according to U.S. Pat. No. 5,226,423, section 7 is about 10-100 mm, section 8 is about 1000-2000 mm, section 9 is about 200-400 mm, section 10 is about 1-5 mm, and section 11 is about 10-50 mm. The diameter of the sensor and guide wire assembly varies between 0.25-2 mm; for use in coronary arteries, the diameter is normally 0.35 mm.

The core wire 1, which extends along the entire length of the sensor and guide wire assembly, is preferably made from a metal, such as stainless steel, or a superelastic metal, e.g. Nitinol®. As should be apparent from FIG. 1 and as is generally known in the art, the mechanical properties (e.g. flexibility and strength) of the sensor guide wire will mainly be determined by the material, design and dimensions of the core wire. In order to enhance the possibility to control the mechanical characteristics of a sensor guide wire, the core wire in each of the sections 7-11 can therefore be given a separate thickness. As was mentioned above, the U.S. Pat. No. 5,226,423 discloses a sensor guide wire with a core wire having a plurality of sections where each section has a different thickness and thereby a different flexibility.

FIG. 2 illustrates schematically a core wire 20, the dimensions of which are the subject of the present invention. The core wire 20 has been divided into seven sections or portions, A-G, where section A is the most proximal section and section G is the most distal section.

The sections relevant for the present patent specification are, where appropriate, characterized by a length and a diameter, where the diameter is taken at the proximal beginning of the section in question. So, for example, section B has a diameter φB and a length LB, section C a diameter φC and a length LC, section D a diameter φD and a length LD, etc.

The core wire 20 illustrated in FIG. 2 is intended to be used in a sensor and guide wire assembly like the one illustrated in FIG. 1, and is in FIG. 2 shown in a state before assembling and mounting of parts such as the sensor element, the signal transmitting cable(s), the dome-shaped tip and the coil. Especially the enlarged portion, which according to FIG. 2 consists of sections C, D and E, has not yet been provided with a recess for reception of a sensor element. Such a recess or depression in the enlarged portion can be made by spark machining, while the enlarged portion itself can be made by removing material from a metal wire having the nominal diameter of the enlarged portion so as to form the smaller diameter portions of the core wire extending distally and proximally of the enlarged portion.

The lengths and especially the diameters of the different sections A-G will be discussed in more detail below, but here it can be mentioned that section B is (in length) about 0-5 mm, section C about 0-1 mm, section D about 0-4 mm, section E about 0-1 mm, and section F about 0-30 mm. It should therefore be noted that the sections A-G of FIG. 2 have no direct relationship to the sections 7-11 of FIG. 1. Further, the subject of the present invention is the dimensions of the sections B-F in general and the diameters φB, φC, 100 F and φG in particular.

A comparison between the lengths of the sections 7-11 of FIG. 1 and the lengths of the sections B-F of FIG. 2 reveals that the present invention is directed to a restricted portion of a core wire in the vicinity of the enlarged portion that is going to accommodate a sensor clement.

From a mechanical (i.e. bending) point of view, the enlarged portion of a core wire can be regarded as stiff, and the object of the present invention is to find suitable dimensions for the portions distally and proximally of this enlarged portion. A sensor guide wire having a core wire with such suitable dimensions should be stiff enough to be pushed forward in narrow and tortuous vessels and yet be flexible enough for manoeuvring into acute takeoffs. The core wire must, at the same time, have such strength so that the performance regarding ability to withstand breakage during operation is further improved.

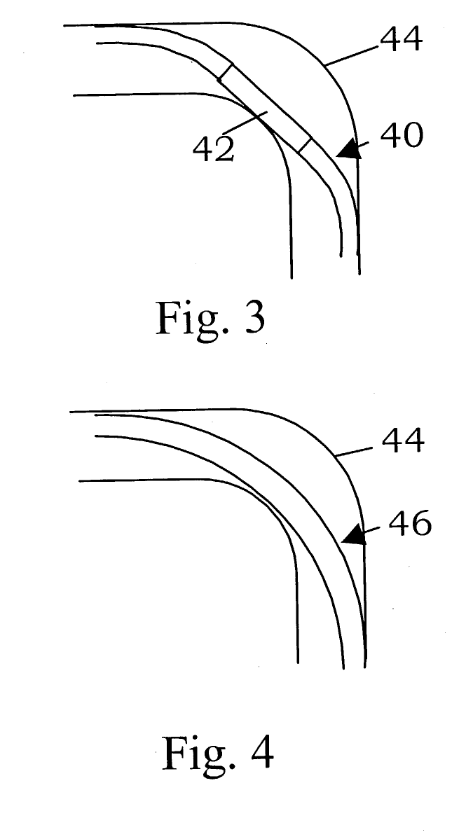

As was mentioned above, it has according to the present invention been found that the strength of a core wire having the general configuration shown in FIG. 2, with a stiff and relatively short enlarged portion, is surprisingly sensitive to the diameters of the portions proximally and distally of this enlarged portion. This fact can be qualitatively understood from an inspection of FIG. 3, which illustrates, in an experimental set-up, how a core wire 40, which includes a portion 42 that can be regarded as stiff, is manoeuvred through a sharp 90-degree bend 44. From the figure one can imagine that the portions distally and proximally of the stiff portion will experience a large strain when the stiff portion is pushed through the sharp bend. It may also be imagined that the strain is concentrated to rather restricted areas, something that increases the risk of having a break of the core wire in these restricted areas.

As a comparison, FIG. 4 illustrates, also in an experimental set-up, how a similar wire 46, which is not provided with a stiff portion, is manoeuvred through the same 90-degree bend 44. In the latter case, the strain exerted on the wire is distributed over large areas, and there are no obvious sites with increased risks of having a break. Here it should be emphasized that the situations illustrated in FIGS. 3 and 4 are not intended to imitate situations known to actually prevail during introduction of a guide wire in a vessel. On the contrary, the situations illustrated in FIG. 3 and FIG. 4 should instead be regarded as part of the analysis leading to the present invention.

By means of experimental set-ups, five different core wire designs were tested, but before presenting the results from these tests in detail it can be mentioned that in the tests the core wires consisted of solid metal wires, whose portions distally and proximally of the enlarged portion exhibited circular cross-sections, the diameters of which were varied. With these prerequisites, the conclusion from the tests is that the diameters of the portions proximally and distally of the enlarged portion should be the same. Here, one has, however, to remember that what actually was varied was the bending resistance, and if the portions proximally and distally of the enlarged portion would have consisted of different materials, with different mechanical properties, or of the same material but with different cross-section shapes, the corresponding conclusion would have been that the bending resistance of the portions proximally and distally of the enlarged portion should be the same. Another way to express this statement is that if the enlarged portion, which according to FIG. 2 consists of sections C, D and E, is removed and the adjoining portions, i.e. sections B and F, are connected to each other, the joint should be as smooth as possible, i.e. the bending resistance should exhibit a continuous behaviour.

As was mentioned above, the enlarged portion of a core wire can be regarded as stiff in comparison with the smaller dimension portions. This means that the specific shape of the enlarged portion does not significantly influence the overall mechanical characteristics of the core wire, which, in turn, implies that, for example, the tapered or conical portions (i.e. portions C and E in FIG. 2) of the enlarged portion can be divided into several conical or tapered portions.

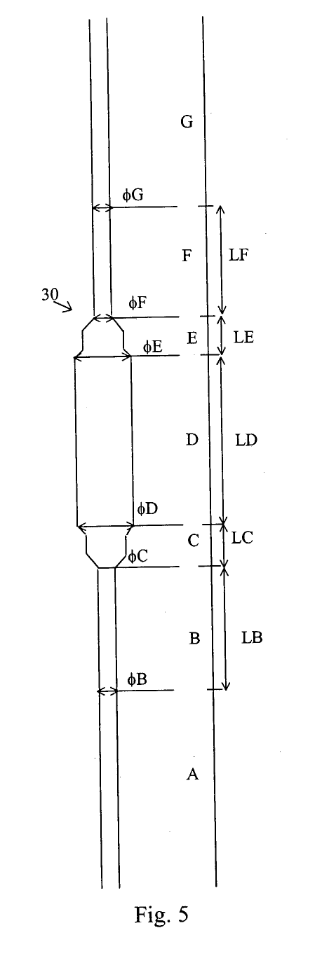

FIG. 5 illustrates a second preferred embodiment of a core wire 30, and a comparison between FIG. 2 and FIG. 5 shows that the design of the core wire 30 differs from the core wire 20 illustrated in FIG. 2 in that the tapered portions C and E of the core wire 20 of FIG. 2 have been further divided into additional portions in the core wire 30 of FIG. 5. However, for the purpose of the present patent specification, also the core wire 30 of FIG. 5 can be regarded as having the same general design as the core wire 20 of FIG. 2, which is reflected by the fact that also the core wire 30 has been divided into seven sections or portions, A-G, where section A is the most proximal section and section G is the most distal section.

Thus, the present invention relates to a core wire for a sensor guide wire assembly for intravascular measurements of physiological variables in a living body. The core wire (20, 30) is provided with different longitudinal sections each having a section diameter (φA-φG), and comprises an enlarged portion, where a sensor is adapted to be arranged, including a predetermined number of sections (C, D, E), one or many distal sections (F, G) positioned distally said enlarged portion and one or many proximal sections (A, B) positioned proximally said enlarged portion, wherein at least one of the sections of said enlarged portion has a larger diameter compared to the diameters of the distal and proximal sections. The core wire is characterized in that the ratio between the diameters (or cross sectional areas, if the core wires are not circular in cross section) (φF, φC) adjacent the enlarged portion is close to 1, preferably between 0.95 and 1.05.

Alternatively this may be expressed as the relative absolute value difference between the diameters of the sections closest to the enlarged portion is less than 5%.

Preferably the diameters of the core wire adjacent to the enlarged portion are equal, i.e. φC equals φF.

The sections E and C may be conically shaped as shown in FIG. 2 or the diameters in sections E and C may increase continuously step-wise as illustrated in FIG. 5.

A typical interval for φC and φF is in the interval 90-130 μm and the largest diameter of the enlarged section is 250-400 μm.

As illustrated in FIG. 2 and FIG. 5 the core wire is symmetrically shaped distally and proximally the enlarged portion. Specifically with regard to the diameters of the core wire, which are such that φC equals φF and φB equals φG, where φB and φG are taken at the same distance from the respective ends of the enlarged portion. Here, the symmetry relates to restricted sections of the core wire distally and proximally of the enlarged portion, i.e. in FIGS. 2 and 5 the section B and section F, respectively. The relevant lengths (from a symmetry point of view) of these sections depend on the diameter of the enlarged portion of the core wire, but can be taken as less than 50 times the diameter of the enlarged portion.

Here it can be mentioned that although it is apparent from the example below that a core wire should be symmetrically shaped distally and proximally the enlarged portion in order to obtain maximal bending strength, other considerations can be involved in the design of a sensor and guide wire assembly, of which a core wire is one part. If, for example, the core wire, despite the measures described herein, should break such that the sensor guide is held together only by one or several coils, it may be advantageous that this break is located distally of the enlarged portion rather than proximally of the enlarged portion. The reason for this is that it may be easier to successfully retract the sensor guide wire in one piece if the retraction is not obstructed by the comparatively large and stiff enlarged portion, which can get stuck in small and tortuous vessels. For a particular design of a sensor and guide wire assembly, a suitable choice may therefore be that the diameters adjacent the enlarged portion are equal, whereas the diameters at a distance proximally and distally of the enlarged portion differ slightly such that the proximal diameter is larger than the distal diameter. In FIGS. 2 and 5, this means that φC=φF and φB>φG, where φB and φG are taken at the same distance from the respective ends of the enlarged portion. The diameter φB can be about 25% larger than the diameter φG. Here it should be noted that even for a sensor guide wire whose final design includes a non-symmetrical core wire, according to the findings of the present invention, a symmetrical core wire would also in this case be the starting point for the design and construction work; and the present invention therefore provides an important contribution to the state of the art also in this case.

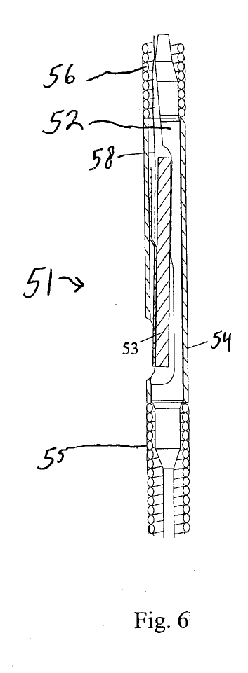

The present invention also relates to a sensor guide wire assembly for intravascular measurements of physiological variables in a living body. An example of such a sensor guide wire assembly is shown in FIG. 6, where a sensor guide 51 comprises a core wire 52, which has been designed according to the teachings above and which extends along substantially the entire length of the sensor guide 51 and has an enlarged portion in which a sensor element 53 is arranged. A jacket or sleeve 54 covers the sensor element 53 and at least a part of the enlarged portion of the core wire 52. At the distal end of this enlarged portion a first coil 55 is provided, while a second coil 56 is provided at the proximal end of the enlarged portion. The core wire 52 is partly disposed inside a flexible tube (not shown) at the proximal end such that the proximal end of the second coil 56 is attached to the distal end of the flexible tube. A sensor guide wire assembly comprises further an electronic unit (not shown in the figure) and a signal transmitting cable 58, which connects the sensor element 53 to the electronic unit and which is disposed inside the second coil 56 and the flexible tube.

Although the present invention has been described with reference to specific embodiments, also shown in the appended drawings, it will be apparent for those skilled in the art that many variations and modifications can be done within the scope of the invention as described in the specification and defined with reference to the claims below. In particular it can be said that the enlarged portion of the core wire, although in all cases being thick and stiff in comparison to its adjacent portions, can have a diameter that ranges from about three quarters of the diameter of the guide wire to the diameter of the guide wire, the last case being when no parts like coils, jackets or sleeves are provided around the enlarged portion.

Thus, in a more generalized description of the present invention a rigid portion of the guide wire is arranged adapted to be provided with a sensor. The rigid portion being stiff in comparison to its adjacent portions, a distal portion and a proximal portion. The distal portion and the proximal portion have equal bending resistances close to the rigid portion (bending resistance may be measured by, for example, the amount of force required to bend a member a certain amount, or the amount of bending resulting from a certain force, etc.). This is achieved in accordance with the above described embodiment of the present invention where the ratio between the diameters (or cross-sectional areas) close to the enlarged portion is close to 1, for example within 5% of each other. In the above described embodiment the guide wire preferably is made from one material.

Alternatively, in another embodiment the equal bending resistance capability may also be achieved by appropriate choices of materials for the distal, rigid and proximal portions irrespectively of the diameters of the different portions. The alternative embodiment may be schematically illustrated as in FIG. 3 where the guide wire and the rigid portion are referred to as 40 and 42, respectively. Here, the rigid portion may be made from one material and the distal and proximal portions from another material. Alternatively, the distal and proximal portions may be made from different materials.

Furthermore, the above-mentioned embodiments of the core wire may have numerous different shapes and be made from a number of combinations of materials.

In one variant, the distal sections are made from one material and the proximal section is made from another material. In one specifically advantageous embodiment the distal sections are made from Nitinol® and the proximal sections are made from stainless steel.

The distal sections may also be made from a combination of materials, preferably arranged such that a base material is covered by another material.

The required performance with regard to bending resistance may be achieved by heat-treatment of the core wire.

The distal sections and proximal sections may have different geometrical cross-sections. This may e.g. be such that the proximal sections' cross-section are hollow forming a hollow tubing so that connections to the sensor may be arranged inside said tubing. The proximal sections have then preferably a circular cross-section where the outer hollow tubing also has a circular cross-section which may be concentric in relation to the outer circular periphery.

### EXAMPLE

Five different core wire designs were tested. All of the tested core wires had the general design shown in FIG. 5, but with different diameters for the sections B, C, F and G. For all of the tested core wires, the following dimensions apply:

LB = LF = 2.5 mm
LC = 0.40 mm
LD = 1.85 mm φD = φE = 0.275 mm
LE = 0.55 mm

In Table 1 and Table 2 below, the dimensions, which were specific for the different core wire designs, are given together with the average number of strokes until break. Table 1 shows the results for core wires provided by a first supplier and Table 2 the results for core wires from a second supplier. The number of tested core wires for each design varied between five (5) and ten (10).

TABLE 1
The number of strokes before break for different core wire
designs for a first supplier of core wires.

| Design | φB [mm] | φC [mm] | φF [mm] | φG[mm] | #Strokes |
|---|---|---|---|---|---|
| I | 0.11 | 0.13 | 0.09 | 0.09 | 264 |
| II | 0.11 | 0.13 | 0.12 | 0.11 | 875 |
| III | 0.11 | 0.13 | 0.12 | 0.09 | 515 |
| IV | 0.09 | 0.11 | 0.11 | 0.09 | 1530 |
| V | 0.11 | 0.11 | 0.11 | 0.09 | 910 |

TABLE 2
The number of strokes before break for different core wire designs for a
second supplier of core wires.

| Design | φB [mm] | φC [mm] | φ [mm]F | φ [mm]G | #Strokes |
|---|---|---|---|---|---|
| I | 0.11 | 0.13 | 0.09 | 0.09 | 326 |
| II | Not | | | | |
| | available | | | | |
| III | 0.11 | 0.13 | 0.12 | 0.09 | 1090 |
| IV | 0.09 | 0.11 | 0.11 | 0.09 | 2305 |
| V | 0.11 | 0.11 | 0.11 | 0.09 | 1450 |

As can be seen from Table 1 and Table 2 for an optimal design regarding the strength of a core wire, the core wire should be as symmetrical as possible around the enlarged portion that is going to accommodate the sensor element. In particular, it is important that diameters of the portions adjacent to the enlarged portion are equal, i.e. φC should be equal to φF. Furthermore, for an optimal design of a core wire, the core wire should be symmetric around the enlarged portion, which here means that φC should be equal to φF and φB should be equal to φG.

What is claimed is:

1. A part for a guide wire assembly, the part comprising:
a core wire having a distal end and a proximal end, the core wire including a rigid portion for supporting a sensor for intravascular measurement of at least one physiological variable, wherein a first portion of the core wire distally from and adjacent to the rigid portion and a second portion of the core wire proximally from and adjacent to the rigid portion have substantially equal bending resistances.

2. A part as set forth in claim 1, wherein the first portion and the second portion are made of different materials.

3. A part as set forth in claim 1, wherein a bending resistance of the first portion is within 5% of a bending resistance of the second portion.

4. A part for a guide wire assembly, the part comprising:
a core wire having a distal end and a proximal end, the core wire including a rigid portion for supporting a sensor for intravascular measurement of at least one physiological variable, wherein a first portion of the core wire distally from and adjacent to the rigid portion and a second portion of the core wire proximally from and adjacent to the rigid portion have bending resistances within 16% of each other.

5. A part for a guide wire assembly, the part comprising:
a core wire having a distal end and a proximal end, the core wire including a rigid portion for supporting a sensor for intravascular measurement of at least one physiological variable, wherein a first portion of the core wire distally from and adjacent to the rigid portion and a second portion of the core wire proximally from and adjacent to the rigid portion have bending resistances within 5% of each other.

6. A part for a guide wire assembly, the part comprising:
a core wire having a distal end and a proximal end, the core wire including a rigid portion for supporting a sensor for intravascular measurement of at least one physiological variable, wherein a first portion of the core wire distally from and adjacent to the rigid portion and a second portion of the core wire proximally from and adjacent to the rigid portion have bending resistances within 10% of each other.

7. A core wire for a sensor guide wire assembly for intravascular measurements of at least one physiological variable in a living body, the core wire provided with different longitudinal sections each having a section diameter, and comprises an enlarged portion, where a sensor is to be arranged, including a predetermined number of sections, at least one distal section positioned distally said enlarged portion and at least one proximal section positioned proximally said enlarged portion, wherein the sections of said enlarged portion have larger diameters compared to the diameters of the distal and proximal sections, wherein the ratio between the diameters adjacent the enlarged portion is close to 1.

8. A core wire according to claim 7, wherein said ratio is between 0.95 and 1.05.

9. A core wire according to claim 7, wherein the relative absolute value difference between the diameters of the sections closest the enlarged portion is less than 5%.

10. A core wire according to claim 7, wherein diameters of the core wire adjacent to the enlarged portion are equal.

11. A core wire according to claim 7, wherein the core wire is symmetrically shaped distally and proximally of the enlarged portion.

12. A core wire according to claim 7, wherein the core wire is symmetrical proximal of the enlarged portion with respect to the core wire distal of the enlarged portion for a predetermined distance.

13. A core wire according to claim 7, wherein at least two sections of the enlarged portion are conically shaped.

14. A core wire according to claim 7, wherein at least two sections of the enlarged portion increase in diameter continuously step-wise.

15. A core wire according to claim 7, wherein said diameters adjacent the enlarged portion are in the interval 90-130 μm.

16. A core wire according to claim 7, wherein the largest diameter of the enlarged portion is 250-400 μm.

17. A core wire for a sensor guide wire assembly for intravascular measurements of at least one physiological variable in a living body, the core wire provided with different longitudinal sections and comprises a rigid portion, where a sensor is to be arranged, at least one distal section positioned distally said rigid portion and at least one proximal section positioned proximally said rigid portion, wherein the ratio between the bending resistances of the sections adjacent said rigid portion is close to 1, and wherein a distal section is made from a one material and a proximal section is made from another material.

18. A core wire according to claim 17, wherein said ratio is between 0.95 and 1.05.

19. A core wire according to claim 17, wherein the core wire is made of a metal or a superelastic metal.

20. A core wire according to claim 17, wherein the rigid portion is made of the same material as either a distal section or a proximal section.

21. A core wire according to claim 17, wherein a distal section is made from Nitinol® and a proximal section is made from stainless steel.

22. A core wire according to claim 17, wherein parts of said core wire are heat-treated.

23. A core wire according to claim 17, wherein distal sections are made from a combination of materials, arranged such that a base material is covered by another material.

24. A core wire according to claim 17, wherein distal sections are arranged such that a base material is covered by another material.

25. A core wire according to claim 17, wherein distal sections and proximal sections have different geometrical cross-sections.

26. A core wire according to claim 17, wherein a proximal cross-section is hollow such that connections to the sensor may be arranged inside and along the section.

27. A sensor guide wire assembly for intravascular measurements of at least one physiological variable in a living body comprising a sensor element, an electronic unit, a signal transmitting cable connecting the sensor element to the electronic unit, a flexible tube having the cable and the sensor element disposed therein, wherein said assembly further comprises a core wire according to claim 7 that is disposed inside the sensor guide wire assembly and extends along substantially the entire length of the sensor guide wire assembly inside the flexible tube.

28. A sensor guide wire assembly according to claim 27, wherein a first coil is attached to a distal end of the enlarged portion.

29. A sensor guide wire assembly according to claim 27, wherein said assembly further comprises a second coil attached to a proximal end of the enlarged portion.

30. A sensor guide wire assembly for intravascular measurements of at least one physiological variable in a living body comprising a sensor element, an electronic unit, a signal transmitting cable connecting the sensor element to the electronic unit, a flexible tube having the cable and the sensor element disposed therein, wherein said assembly further comprises a part according to claim 1 that is disposed inside the sensor guide wire assembly and extends along substantially the entire length of the sensor guide wire assembly inside the flexible tube.
